(19) United States
(12) Patent Application Publication
(10) Pub. No.: US 2005/0070792 A1
(43) Pub. Date: Mar. 31, 2005

(54) Therapeutic imaging system, and radiation therapeutic imaging system and method of controlling it

(76) Inventors: Akira Mizukoshi, Tokyo (JP); Toshihiro Sakuma, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 10/945,711

(22) Filed: Sep. 21, 2004

(30) Foreign Application Priority Data
Sep. 26, 2003 (JP) 2003-335735

(51) Int. Cl. A61B 6/00

(52) U.S. Cl. 600/427; 378/20; 378/62; 378/65

(57) ABSTRACT

A therapeutic imaging system includes a first therapeutic/imaging apparatus for performing at least one of therapy and imaging, a second therapeutic/imaging apparatus for use in combination with the first therapeutic/imaging apparatus for performing at least one of therapy and imaging, a first carriage device for laving thereon a subject, for use in combination with at least one of the first and second therapeutic/imaging apparatuses, a second carriage device for laying thereon a subject, for use in combination with the second therapeutic/imaging apparatus, spaced apart from the first carriage device at a certain distance, and a moving device disposed between the first and second carriage devices, wherein the first and second carriage devices are concatenately disposed, and the moving device moves the second therapeutic/imaging apparatus on a straight line connecting the first and second carriage devices.

### BACKGROUND OF THE INVENTION

The present invention relates to a therapeutic imaging system, and a radiation therapeutic imaging system and a method of controlling it, and particularly to a therapeutic imaging system, and a radiation therapeutic imaging system and a method of controlling it for operating a plurality of therapeutic/imaging apparatuses in combination or separately for performing at least one of therapy and imaging.

In the medical field, there is known a technique of using a plurality of apparatuses in combination to perform imaging and therapy. For example, one such process involves conducting vascular imaging by X-rays, making transcutaneous treatment, and conducting tomographic imaging for examination on a region around that vascular-imaged. Such an application of radiation imaging technique to therapy is sometimes referred to as IVR (interventional radiology).

A known apparatus for the tomographic imaging is, for example, the X-ray CT (computed tomography) apparatus, which emits X-rays as radiation, detects X-rays passing through a subject, and computationally produces a tomographic image.

Specifically, the X-ray CT apparatus has an X-ray source and an X-ray detector facing the X-ray source over the subject in a scan gantry. The scan gantry rotates around the subject while emitting X-rays from the X-ray source and detecting X-rays passing through the subject by the X-ray detector in a plurality of directions with respect to the subject, and a tomographic image is produced based on the projection data. This produces a tomographic image in a plane perpendicular to the scan direction. During this, the subject is placed on carriage means for imaging. The carriage means for imaging will be sometimes referred to as a cradle hereinbelow.

A known apparatus for vascular imaging is, for example, the angiography apparatus that has an X-ray source and an X-ray detector facing the X-ray source over the subject in a scan gantry, as in the X-ray CT apparatus. The angiography apparatus will be sometimes referred to as the Angio apparatus hereinbelow. The angiography apparatus emits X-rays toward a subject injected with a contrast agent at a predefined region to image blood vessels. The subject here is also placed on a cradle for imaging.

A system for IVR in which the therapeutic and imaging apparatuses are used in combination is known as a therapeutic imaging system, in which an X-ray CT apparatus, a vascular imaging apparatus, and a cradle common to these apparatuses are installed in one room. The cradle is substantially fixed and is finely adjusted to a predefined imaging region when imaging.

In vascular imaging, the angiography apparatus moves the X-ray source and X-ray detector to image the subject on the cradle. At that time, the scan gantry of the X-ray CT apparatus is moved away to an area in the room sufficiently spaced apart from the vascular imaging apparatus to avoid interference with the vascular imaging apparatus, i.e., to an area outside of the range of motion of the angiography apparatus.

On the other hand, in tomographic imaging by the X-ray CT apparatus, the cradle allows the subject's position to be finely adjusted to an imaging field of view of the X-ray CT apparatus, and the X-ray CT apparatus moves the scan gantry to conduct imaging. At that time, the scan gantry of the angiography apparatus is moved away to an area sufficiently spaced apart from the X-ray CT apparatus to avoid interference with the X-ray CT apparatus.

Moreover, as such a vascular imaging/CT apparatus, there is known one comprising a CT gantry, a vascular imaging arm, a vascular imaging top plate for laying thereon a subject of vascular imaging, a CT imaging top plate for laying thereon a subject of CT imaging, and moving means for moving the vascular imaging top plate, CT imaging top plate, CT gantry or vascular imaging arm, thereby effectively using the CT gantry and vascular imaging arm and improving the efficiency of examination (for example, see Patent Document 1).
[Patent Document 1] Japanese Patent Application Laid Open No. 2001-190535.

In the conventional therapeutic imaging system, however, when the CT gantry is to be moved to a next room, it is moved in a direction perpendicular to the scan direction, i.e., in the longitudinal direction of the CT gantry. As a result, the distance of travel of the CT gantry is at least longer than its longitudinal dimension. This increases the length of the wiring cables attached to the CT gantry and therefore requires that attention be given to the longer wiring cables during movement over a long distance. Moreover, the track on which the CT gantry is moved is complex, so that it is difficult to secure safety in the travel direction.

### SUMMARY OF THE INVENTION

Therefore, an object of the present invention is to provide a therapeutic imaging system, and a radiation therapeutic imaging system and a method of controlling it using a plurality of therapeutic/imaging apparatuses in combination or separately, in which the distance of travel of one of the therapeutic/imaging apparatuses is reduced and safety securance in the travel direction is facilitated.

To attain the aforementioned objects, a therapeutic imaging system in accordance with the present invention comprises: a first therapeutic/imaging apparatus for performing at least one of therapy and imaging; a second therapeutic/imaging apparatus for use in combination with the first therapeutic/imaging apparatus for performing at least one of therapy and imaging; first carriage means for use in combination with at least one of the first and second therapeutic/imaging apparatuses; second carriage means for use in combination with the second therapeutic/imaging apparatus, spaced apart from the first carriage means at a certain distance; and moving means disposed between the first and second carriage means. The first and second carriage means are concatenately disposed, and the moving means moves the second therapeutic/imaging apparatus on a straight line connecting the first and second carriage means.

According to the therapeutic imaging system of the present invention, the first and second carriage means are concatenately disposed so that their longitudinal dimensions are aligned on a straight line. The second therapeutic/imaging apparatus is moved between the first and second carriage means along the moving means.

To attain the aforementioned objects, a radiation therapeutic imaging system in accordance with the present invention comprises: a first radiation therapeutic/imaging apparatus for performing at least one of therapy and imaging by emitting radiation; a second radiation therapeutic/imaging apparatus for use in combination with the first radiation therapeutic/imaging apparatus for performing at least one of therapy and imaging by emitting radiation; first carriage means for use in at least one of the first and second radiation therapeutic/imaging apparatuses; second carriage means for use in combination with the second radiation therapeutic/imaging apparatus, spaced apart from the first carriage means at a certain distance; moving means disposed between the first and second carriage means; and an open/close door provided between the first and second carriage means for separating a region in which the first and second carriage means are installed into two sub-regions. The first and second carriage means are concatenately disposed, and the moving means moves the second radiation therapeutic/imaging apparatus on a straight line connecting the first and second carriage means.

According to the radiation therapeutic imaging system of the present invention, the first and second carriage means are concatenately disposed so that their longitudinal dimensions are aligned on a straight line. The second therapeutic/imaging apparatus is moved between the first and second carriage means along the moving means. Furthermore, the first and second carriage means are separated in different sub-regions by the door.

To attain the aforementioned objects, a method of controlling a radiation therapeutic imaging system in accordance with the present invention is for use in a radiation imaging system comprising: a first radiation therapeutic/imaging apparatus for performing at least one of therapy and imaging by radiation; a second therapeutic/imaging apparatus that is movable, for use in combination with the first radiation therapeutic/imaging apparatus for performing at least one of therapy and imaging by radiation; first carriage means for use in at least one of the first and second radiation therapeutic/imaging apparatuses; second carriage means for use in combination with the second radiation therapeutic/imaging apparatus, spaced apart from the first carriage means at a certain distance and disposed concatenately with the first carriage means; moving means disposed between the first and second carriage means for moving the second therapeutic/imaging apparatus on a straight line connecting the first and second carriage means; and an open/close door provided between the first and second carriage means for separating a region in which the first and second carriage means are installed into two sub-regions, and the method comprises moving the first radiation therapeutic/imaging apparatus, second radiation therapeutic/imaging apparatus, first carriage means and second carriage means relative to one another, and in a first condition, comprises the steps of: performing therapy or imaging at a predefined position by the second radiation therapeutic/imaging apparatus with the first radiation therapeutic/imaging apparatus fixed, while monitoring positions of at least the second radiation therapeutic/imaging apparatus and first carriage means; and performing therapy or imaging at a predefined position by the first radiation therapeutic/imaging apparatus with the second radiation therapeutic/imaging apparatus fixed, while monitoring positions of at least the first radiation therapeutic/imaging apparatus and first carriage means; and in a second condition, performing therapy or imaging at a predefined position by the first radiation therapeutic/imaging apparatus while monitoring positions of the first radiation therapeutic/imaging apparatus and first carriage means; and performing therapy or imaging at a predefined position by the second radiation therapeutic/imaging apparatus while monitoring positions of the second radiation therapeutic/imaging apparatus and second carriage means, wherein in the second condition, the method separately conducts the step of performing therapy or imaging by the first radiation therapeutic/imaging apparatus and the step of performing therapy or imaging by the second radiation therapeutic/imaging apparatus.

According to the method of controlling a radiation therapeutic imaging system of the present invention, in a first condition, therapy or imaging is performed at a predefined position by the second radiation therapeutic/imaging apparatus with the first radiation therapeutic/imaging apparatus fixed, while monitoring positions of at least the second radiation therapeutic/imaging apparatus and first carriage means. Moreover, therapy or imaging is performed at a predefined position by the first radiation therapeutic/imaging apparatus with the second radiation therapeutic/imaging apparatus fixed, while monitoring positions of at least the first radiation therapeutic/imaging apparatus and first carriage means.

In a second condition, therapy or imaging is performed at a predefined position by the first radiation therapeutic/imaging apparatus while monitoring positions of the first radiation therapeutic/imaging apparatus and first carriage means. Moreover, therapy or imaging is performed at a predefined position by the second radiation therapeutic/imaging apparatus while monitoring positions of the second radiation therapeutic/imaging apparatus and second carriage means.

In the second condition, the therapy or imaging by the first radiation therapeutic/imaging apparatus, and that by the second radiation therapeutic/imaging apparatus are separately conducted.

According to the therapeutic imaging system of the present invention, a plurality of therapeutic/imaging apparatuses are used in combination or separately, in which the distance of travel of one of the therapeutic/imaging apparatuses is reduced and safety securance in the travel direction is facilitated.

According to the radiation therapeutic imaging system of the present invention, a plurality of therapeutic/imaging apparatuses are used in combination or separately, in which the distance of travel of one of the therapeutic/imaging apparatuses is reduced and safety securance in the travel direction is facilitated.

According to the method of controlling a radiation therapeutic imaging system of the present invention, a plurality of therapeutic/imaging apparatuses are used in combination or separately, in which the distance of travel of one of the therapeutic/imaging apparatuses is reduced and safety securance in the travel direction is facilitated.

Further objects and advantages of the present invention will be apparent from the following description of the preferred embodiments of the invention as illustrated in the accompanying drawings.

### BRIEF DESCRIPTION OF THE DRAWINGS

FIG. 1 is a functional block diagram schematically showing the function of a radiation therapeutic imaging system in accordance with one embodiment of the present invention.

FIG. 2 is a schematic top view generally showing the radiation therapeutic imaging system in the first state in accordance with a first embodiment of the present invention.

FIG. 3 is a schematic side view generally showing the radiation therapeutic imaging system shown in FIG. 2.

FIG. 4 is a flow chart showing the process of shifting from the first state to the second state in the radiation therapeutic imaging system in accordance with the first embodiment of the present invention.

FIG. 5 is a flow chart showing the process of shifting from the first state to the second state in the radiation therapeutic imaging system in accordance with the first embodiment of the present invention.

FIG. 6 is a flow chart showing the process of shifting from the first state to the second state in the radiation therapeutic imaging system in accordance with the first embodiment of the present invention.

FIG. 7 is a schematic top view generally showing the radiation therapeutic imaging system in the second state in accordance with the first embodiment of the present invention.

FIG. 8 is a schematic side view generally showing the radiation therapeutic imaging system shown in FIG. 7.

FIG. 9 is a flow chart showing the process of shifting from the second state to the first state in the radiation therapeutic imaging system in accordance with the first embodiment of the present invention.

FIG. 10 is a flow chart showing the process of shifting from the second state to the first state in the radiation therapeutic imaging system in accordance with the first embodiment of the present invention.

FIG. 11 is a flow chart showing the process of shifting from the second state to the first state in the radiation therapeutic imaging system in accordance with the first embodiment of the present invention.

FIG. 12 is a schematic side view generally showing the radiation therapeutic imaging system in the second state in accordance with a second embodiment of the present invention.

FIG. 13 is a schematic side view generally showing the radiation therapeutic imaging system in the second state in accordance with the second embodiment of the present invention.

FIG. 14 is a schematic side view generally showing the radiation therapeutic imaging system in the first state in accordance with a third embodiment of the present invention.

FIG. 15 is a schematic side view generally showing the radiation therapeutic imaging system in the second state in accordance with the third embodiment of the present invention.

### DETAILED DESCRIPTION OF THE INVENTION

Best modes for carrying out the present invention will now be described with reference to the accompanying drawings.

### FIRST EMBODIMENT

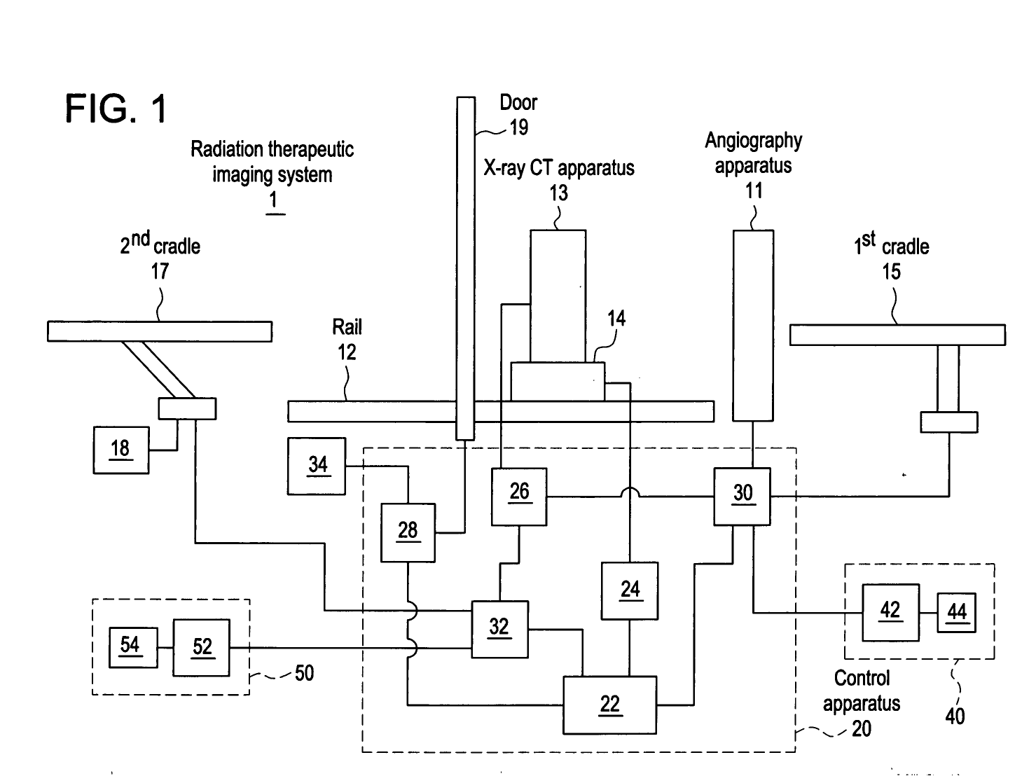

FIG. 1 is a block diagram schematically showing the function of a radiation therapeutic imaging system 1 in accordance with the present embodiment.

The radiation therapeutic imaging system 1 comprises an angiography apparatus 11, an X-ray CT apparatus 13, a first cradle 15, a second cradle 17, a rail 12, and a door 19, all disposed in one region.

One embodiment of the first radiation therapeutic/imaging apparatus in the present invention corresponds to the angiography apparatus 11. One embodiment of the second radiation therapeutic/imaging apparatus corresponds to the X-ray CT apparatus 13. Moreover, embodiments of the first and second carriage means correspond to the first and second cradles 15 and 17, respectively.

The radiation therapeutic imaging system 1 also comprises a control apparatus 20, an angiography apparatus driving/stopping apparatus 40, an X-ray CT apparatus driving/stopping apparatus 50, a door opening/closing section 34, and a second cradle control switch 18.

One embodiment of the control means in the present invention corresponds to the control apparatus 20. Moreover, one embodiment of the first driving/stopping means in the present invention corresponds to the angiography apparatus driving/stopping apparatus 40, and one embodiment of the second driving/stopping means corresponds to the X-ray CT apparatus driving/stopping apparatus 50.

The angiography apparatus 11 comprises an X-ray source and an X-ray detector facing the X-ray source over a subject laid on the first cradle 15.

The X-ray CT apparatus 13 is secured on a base 14, and is moved along the rail 12 between the first and second cradles 15 and 17 by a motor, for example, which is not shown. The X-ray CT apparatus 13 comprises a scan gantry provided with an X-ray source and an X-ray detector facing the X-ray source over a subject.

The first cradle 15 is disposed near the angiography apparatus 11. The first cradle 15 is one ordinarily used in the angiography apparatus 11.

The first cradle 15 is configured so that its position, height, angle etc. can be finely adjusted by, for example, a central processing section (not shown), for preventing collision and adjusting the imaging position.

The second cradle 17 is disposed to be spaced apart from the first cradle 15 at a certain distance, and disposed concatenately with the first cradle 15. The fact that the longitudinal dimensions of the first and second cradles 15 and 17 are aligned approximately in a straight line is expressed by the term “concatenately.”

The second cradle 17 is used in combination with the X-ray CT apparatus 13, and it is one ordinarily used in the X-ray CT apparatus 13.

The rail 12 is provided on a straight line connecting the first and second cradles 15 and 17.

The door 19 is provided between the first and second cradles 15 and 17. Moreover, the door 19 is openable/closable.

The control apparatus 20 comprises a mode selecting section 22, a base driving section 24, an X-ray CT apparatus selecting section 26, a door monitoring section 28, a first monitoring section 30, and a second monitoring section 32.

The control apparatus 20 monitors and controls the position of the X-ray CT apparatus 13, angiography apparatus 11, and first and second cradles 15 and 17 relative to one another.

The mode selecting section 22 is connected to the base driving section 24, door monitoring section 28, first monitoring section 30 and second monitoring section 32.

The mode selecting section 22 is configured to allow selection between a first state in which the X-ray CT apparatus 13 and angiography apparatus 11 are used in combination, and a second state in which the X-ray CT apparatus 13 and angiography apparatus 11 are operated separately rather than in combination. A human operator operates the mode selecting section 22 to select the first or second state.

The mode selecting section 22 also makes selection between opening and closing of the door 19 according to the position of the base 14 of the X-ray CT apparatus 13 and the selected mode. The mode selecting section 22 is implemented by, for example, a central processing apparatus and software, and can be remotely operated.

The base driving section 24 is connected to the base 14 of the X-ray CT apparatus 13 and mode selecting section 22. The base driving section 24 issues a command to, for example, a motor (not shown) according to the mode selected by the mode selecting section 22 to move the base 14. Moreover, the base driving section 24 provides information on the position of the base 14 to the mode selecting section 22.

The X-ray CT apparatus selecting section 26 selects the first or second monitoring section 30 or 32 according to the mode selected by the mode selecting section 22. As a result, the cradle 15 or 17 corresponding to the X-ray CT apparatus 13 is selected via the first or second monitoring section 30 or 32.

The door monitoring section 28 is connected to the mode selecting section 22 and a door opening/closing apparatus 34. The door monitoring section 28 monitors opening/closing of the door according to the mode selected by the mode selecting section 22 and information on the position.

The first monitoring section 30 is connected to the angiography apparatus 11, first cradle 15, mode selecting section 22, X-ray CT apparatus selecting section 26 and angiography apparatus stopping apparatus 40. The first monitoring section 30 monitors the operation and position of the angiography apparatus 11, X-ray CT apparatus 13 and first cradle 15 according to the mode selected by the mode selecting section 22.

The second monitoring section 32 is connected to the mode selecting section 22, X-ray CT apparatus 13, second cradle 17 and X-ray CT apparatus driving/stopping apparatus 50. The second monitoring section 32 monitors the operation and position of the X-ray CT apparatus 13 and second cradle 17 according to the mode selected by the mode selecting section 22.

The door opening/closing apparatus 34 is connected to the door monitoring section 28. The operator sends a command to open/close the door 19 to the door monitoring section 28 at the door opening/closing apparatus 34.

The angiography apparatus driving/stopping apparatus 40 comprises an angiography apparatus driving section 42 and an angiography apparatus stopping switch 44.

The operator makes selection between driving for imaging and stopping in an emergency at the angiography apparatus driving/stopping apparatus 40. As a result, a signal indicating driving or stopping is sent to the angiography apparatus 11 via the first monitoring section 30. The angiography apparatus driving/stopping apparatus 40 is implemented by a central processing apparatus, for example.

The X-ray CT apparatus driving/stopping apparatus 50 comprises an X-ray CT apparatus driving section 52 and an X-ray CT apparatus stopping switch 54.

The operator makes selection between driving for imaging and stopping in an emergency at the X-ray CT apparatus driving/stopping apparatus 50. As a result, a signal indicating driving or stopping is sent to the X-ray CT apparatus 13 via the second monitoring section 32. The X-ray CT apparatus driving/stopping apparatus 50 is implemented by a central processing apparatus, for example.

The second cradle control switch 18 selects control over the second cradle 17. The operator turns on/off the switch to enable control of the second cradle 17 by the second monitoring section 32.

A process of conducting IVR using the X-ray CT apparatus and angiography apparatus in combination will now be described with reference to FIGS. 1, 2 and 3.

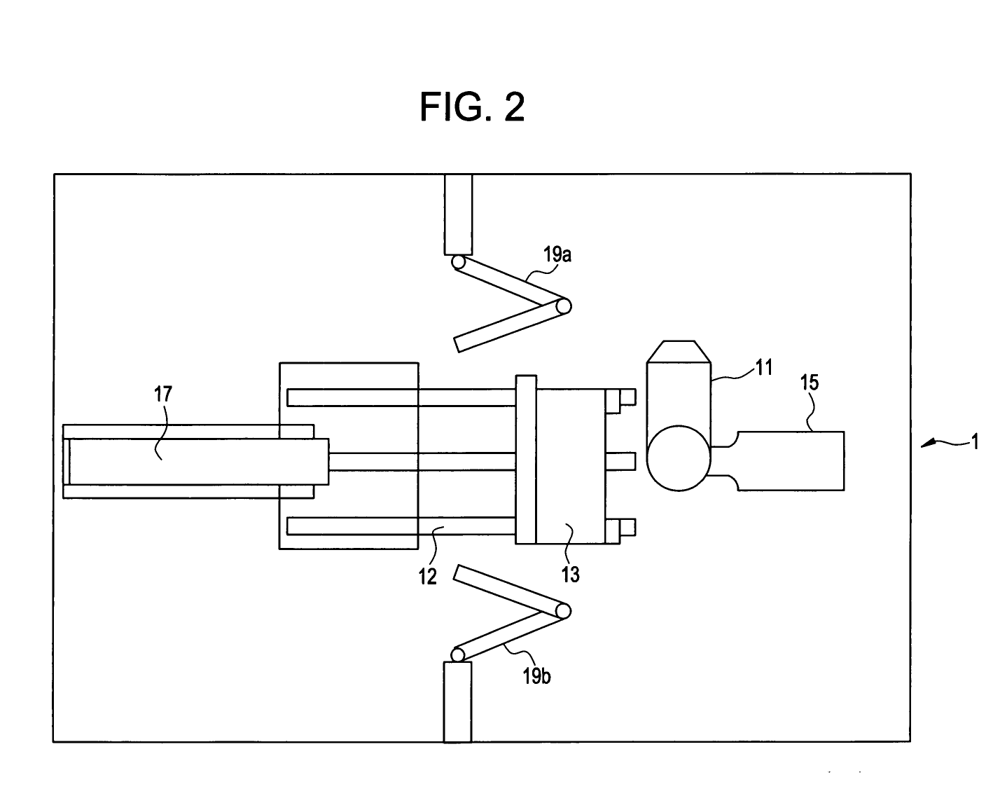

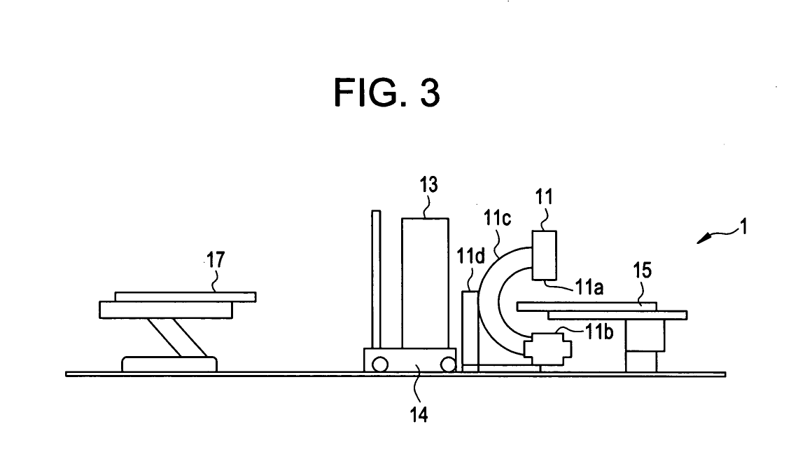

FIG. 2 is a schematic top view generally showing the radiation therapeutic imaging system 1 in accordance with the present embodiment, and FIG. 3 is a schematic side view generally showing the radiation therapeutic imaging system in accordance with the present embodiment. One embodiment of the first state of the present invention corresponds to a state in which IVR is conducted using the X-ray CT apparatus and angiography apparatus in combination.

As shown in FIGS. 2 and 3, in the first state, the angiography apparatus 11 and X-ray CT apparatus 13 are disposed near the first cradle 15. Moreover, the doors 19a and 19b are open.

In the first state using the X-ray CT apparatus 13, angiography apparatus 11 and first cradle 15 in combination, the angiography apparatus 11 is first used to conduct vascular imaging on a certain region in a subject on the first cradle 15. A mode in which imaging is conducted using the angiography apparatus in the first state will be sometimes referred to as an angiographic mode hereinbelow.

The operator selects the angiographic mode in the first state at the mode selecting section 22.

The base driving section 24 first moves the base 14 wheels. As a result, the X-ray CT apparatus 13 is moved away along with the base 14 from the range of motion of the angiography apparatus 11 to a position at which interference with the angiography apparatus 11 is avoided.

Moreover, the X-ray CT apparatus selecting section 26 is set to select the first monitoring section 30. As a result, the first cradle 15 is selected as a cradle whose position is to be adjusted relative to the X-ray CT apparatus 13. Furthermore, the first monitoring section 30 prohibits an operation of the X-ray CT apparatus 13 via the X-ray CT apparatus selecting section 26.

Next, the relative position of the angiography apparatus 11 and first cradle 15 is adjusted. At that time, the first monitoring section 30 monitors the relative distance between the angiography apparatus 11 and first cradle 15. The first monitoring section 30 also intercepts a command from the angiography apparatus driving section 42 when the angiography apparatus 11, X-ray CT apparatus 13 and first cradle 15 come closer than a certain distance to one another.

As shown in FIG. 3, the angiography apparatus 11 is comprised of a curved first support 11c and a second support 11d having one portion fixed to the first support 11c and another portion secured to a floor, and the first support 11c is provided with an X-ray source 11a at its upper end and an X-ray detector 11b at its lower end. The operator operates the angiography apparatus driving/stopping apparatus 40 to rotate the supports 11c and 11d around fixed positions on the supports 11c and 11d, and position the angiography apparatus 11 over a predefined imaging region in the subject on the first cradle 15. After positioning the imaging region, the angiography apparatus 11 conducts imaging on the subject.

Next, in the first state, imaging is shifted from the angiography apparatus 11 to the X-ray CT apparatus 13. Such a condition will be sometimes referred to as a shifting mode.

First, the operator selects the shifting mode in the first state at the mode selecting section 22. At that time, the first monitoring section 30 monitors the relative distance between the first cradle 15 and X-ray CT apparatus 13. In such a condition, the operator moves the angiography apparatus 11 away from the vicinity of the first cradle 15 using the angiography apparatus driving section 42.

The operator next adjusts the position, height, angle etc. of the first cradle 15 to an imaging region of the X-ray CT apparatus 13. The first cradle 15 is adjusted by, for example, the angiography apparatus driving section 42.

Then, imaging is conducted by the X-ray CT apparatus 13. Such a condition will be sometimes referred to as a CT mode.

The operator selects the CT mode in the first state at the mode selecting section 22. The angiography apparatus 11 is then moved by the angiography apparatus driving section 42 away from the range of motion of the X-ray CT apparatus 13 to avoid interference with the X-ray CT apparatus 13, and is prohibited from operating by the first monitoring section 30.

The X-ray CT apparatus 13 and its base 14 are then moved to the vicinity of the first cradle 15 along the rail 12 by the base driving section 24.

Then, the relative position of the X-ray CT apparatus 13 and first cradle 15 is adjusted. At that time, the first monitoring section monitors the relative position of the X-ray CT apparatus 13 and first cradle 15, and intercepts a command from the X-ray CT apparatus driving section 52 when they come closer than a certain distance to each other.

The X-ray CT apparatus 13 conducts tomographic imaging on the subject while rotating the scan gantry of the X-ray CT apparatus 13 about the subject on the cradle around a head-to-toe axis of the subject's body and also translating the scan gantry in the body axis direction.

In such a first state, the angiography apparatus 11, X-ray CT apparatus 13 and first cradle 15 are controlled by the control apparatus 20 by monitoring information on their positions so that they are prevented from colliding with one another and their operation is stopped when they come closer than a certain distance to one another.

Moreover, the control apparatus 20 also controls the angiography apparatus 11 and X-ray CT apparatus 13 to prohibit overlap of their radiation emission. That is, the angiography apparatus 11 and X-ray CT apparatus 13 cannot emit X-rays at the same time.

Furthermore, the control apparatus 20 establishes a setting so that when at least one of the angiography apparatus 11 and X-ray CT apparatus 13 is stopped in an emergency, the other is stopped in synchronization.

In the first state, even if the operator tries to operate the door opening/closing apparatus 34 to close the door 19, the command to open/close the door is intercepted by the door monitoring section 28 and the door 19 is not activated to close.

Moreover, although the apparatuses are operated with the door 19 open in the preceding description, they may be operated with the door 19 closed. When the operation is made with the door 19 closed, the action of the door 19 is monitored by the door monitoring section 28 as above, and the door 19 is not activated to open.

A process of shifting from the first state to the second state will now be described in detail with reference to FIG. 1 and FIGS. 4-6.

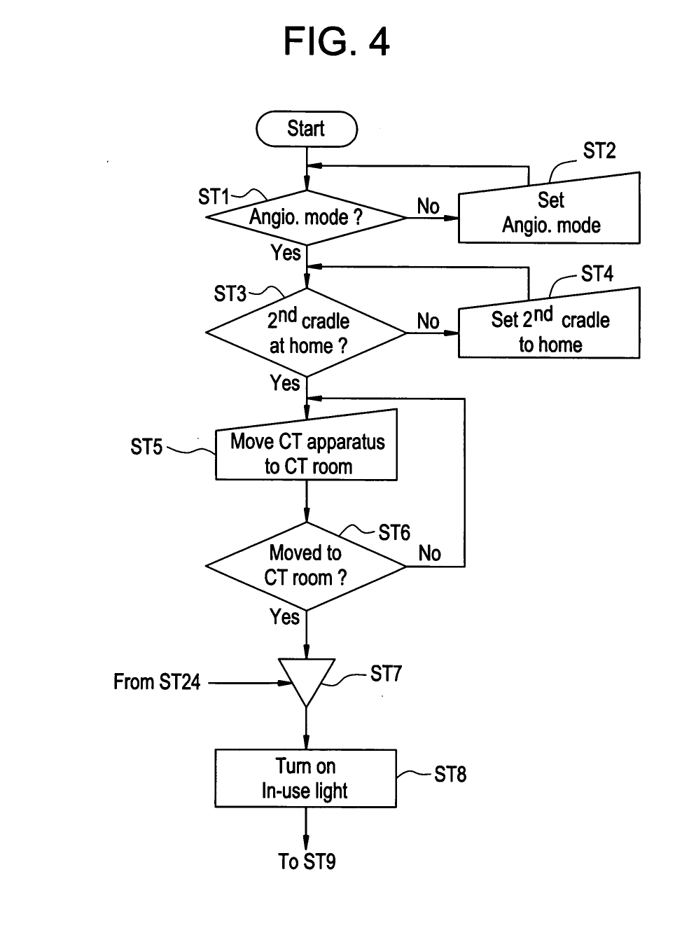

FIGS. 4-6 are flow charts generally showing the process of shifting from the first state to the second state in the radiation therapeutic imaging system 1 in accordance with the present embodiment.

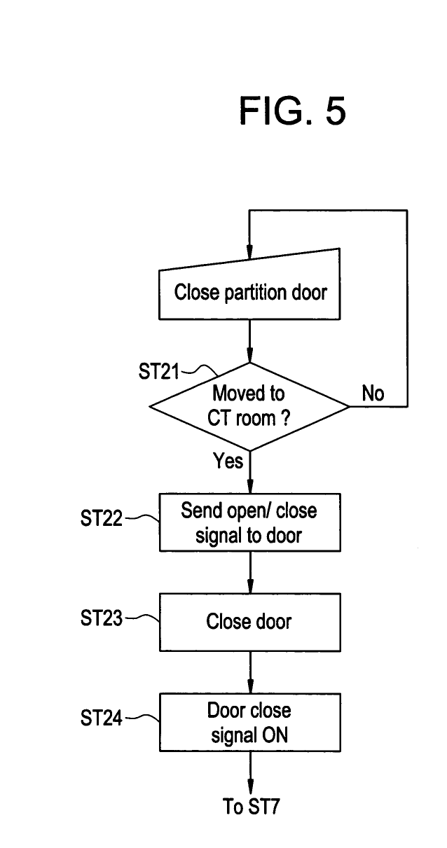

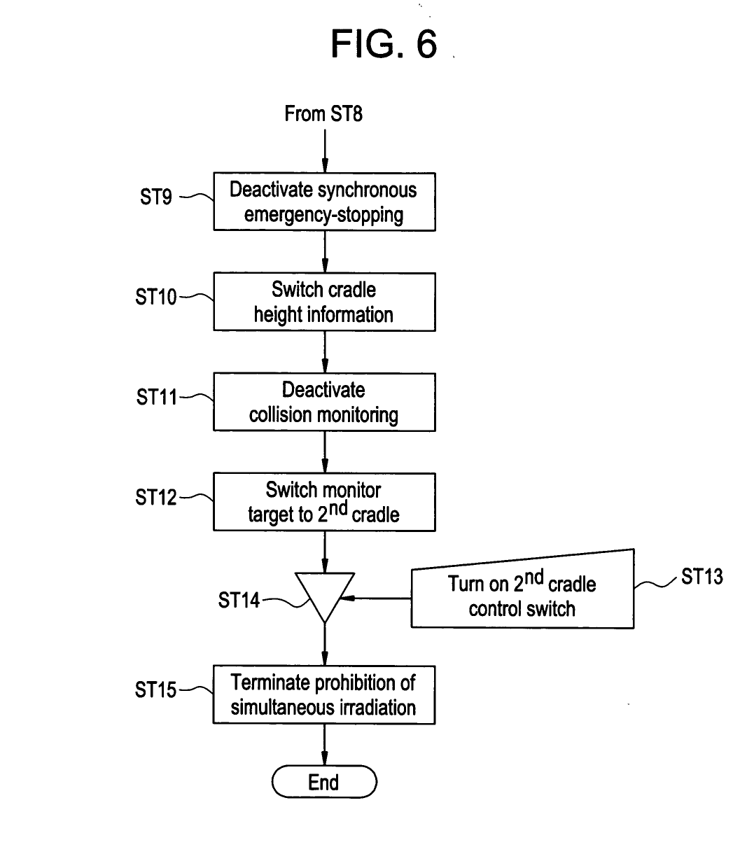

Steps ST1-ST15 shown in FIGS. 4 and 6 are mainly executed by the control apparatus 20, angiography apparatus driving/stopping apparatus 40 and X-ray CT apparatus driving/stopping apparatus 50 shown in FIG. 1, and Steps ST21-ST24 shown in FIG. 5 are mainly executed by the door driving apparatus 34.

First, as shown in FIG. 4, a confirmation is made at the control apparatus 20 that the mode selecting section 22 is in the angiographic mode (ST1). For example, when tomographic imaging by the X-ray CT apparatus 13 in the first state is completed, the mode selecting means 22 is in the CT mode; therefore, the X-ray CT apparatus 13 is moved away from the range of motion of the angiography apparatus 11 along the rail 12, the angiography apparatus 11 and first cradle 15 is disposed at a predefined position, and then the mode is switched to the angiographic mode (ST2).

Next, a confirmation is made at the control apparatus 20 that the second cradle 17 is at a predefined reference position (ST3). If the second cradle 17 is at a position other than the reference position, it is re-disposed at the reference position (ST4). The reference position will be sometimes referred to as the home of the second cradle 17 hereinbelow.

The base driving section 24 is then operated to move the X-ray CT apparatus 13 and base 14 to a CT room along the rail 12 (ST5). As used herein, the term “CT room” refers to an area separated off by the door 19 in which the X-ray CT apparatus 13 and second cradle 17 are disposed in the second state. The X-ray CT apparatus 13 and base 14 are moved on a straight line connecting the first and second cradles along the rail 12 after the angiography apparatus 11 has been moved away from the range of motion of the X-ray CT apparatus 13.

Subsequently the control apparatus 20 makes a confirmation that the X-ray CT apparatus 13 has been moved to a predefined position near the second cradle 17 (ST6). If the X-ray CT apparatus 13 is not moved to the predefined position, the flow again goes back to Step ST5. The predefined position near the second cradle 17 at which the X-ray CT apparatus 13 is to be disposed will be sometimes referred to as a CT position.

On the other hand, as shown in FIG. 5, when the door 19 is to be closed, a confirmation is first made via the door monitoring section 28 and mode selecting section 22 that the X-ray CT apparatus 13 has been moved to the predefined position in the CT room (ST21). If it has not been moved, the flow of operations cannot proceed.

After confirming that the X-ray CT apparatus 13 moved to the CT position, the door opening/closing apparatus 34 sends an open/close signal to the door 19 (ST22).

The door monitoring section 28 then permits the door 19 to be closed, and the door 19 is closed (ST23). Upon closing of the door 19, a signal indicating that the door 19 is closed is output (ST24).

As shown in FIG. 4, once the control apparatus 20 has made confirmation of the two conditions (ST7), i.e., that the X-ray CT apparatus 13 has been moved to the CT position (ST6) and that a signal indicating the door 19 being closed has been output (ST24), lights for indicating that the two rooms separated by the door 19 are in use are turned on (ST8). The two rooms are two regions separated by the door, i.e., the CT room and an angiography room. The lights indicating usage are provided outside the rooms or in an operation room for the angiography apparatus 11 and X-ray CT apparatus 13.

Next, as shown in FIG. 6, the control apparatus 20 deactivates the synchronous emergency-stopping (ST9), because the angiography apparatus 11 and X-ray CT apparatus 13 have been set to be synchronously stopped in an emergency in the first state. As a result, the apparatuses can now be separately stopped in an emergency.

Next, the height of the first cradle 15 is set to be suitable for measurement by the angiography apparatus 11 in the angiography room and the height of the second cradle 17 is set to be suitable for measurement by the X-ray CT apparatus 13 in the CT room (ST10).

The first monitoring section 30 then deactivates monitoring for preventing collision of the angiography apparatus 11 and X-ray CT apparatus 13 (ST11).

The X-ray CT apparatus selecting section 26 then switches monitoring of the X-ray CT apparatus 13 from the first monitoring section 30 to the second monitoring section 32 to monitor the relative position of the X-ray CT apparatus 13 and second cradle 17 (ST12).

On the other hand, the second cradle control switch 18, for example, is activated so that the second cradle 17 can be controlled (ST13). By activating the second cradle control switch 18, an action of the door 19 is prohibited, and consequently, the door is prevented from being accidentally opened.

When the two conditions that the relative position of the X-ray CT apparatus 13 and second cradle 17 can be monitored (ST12) and that the second cradle control switch 18 is activated (ST13) are confirmed (ST14), the prohibition of overlap of radiation emission by the angiography apparatus 11 and X-ray CT apparatus 13 is terminated (ST15). That is, prohibition of simultaneous irradiation is terminated.

By the process as described above, the first state is shifted to the second state.

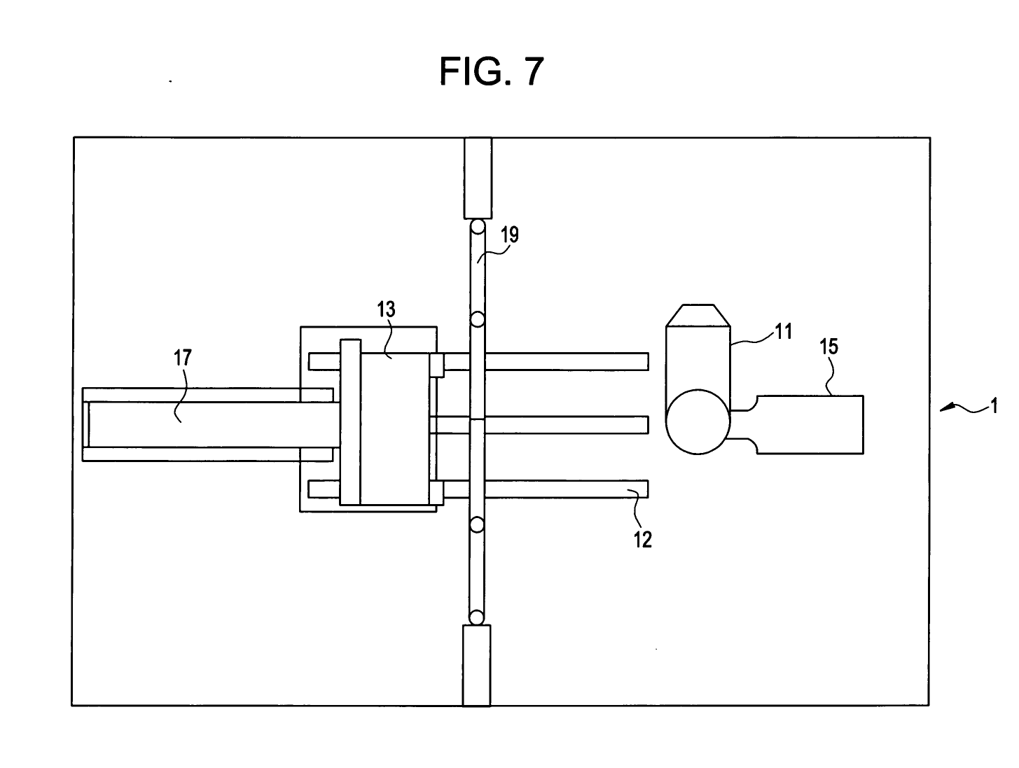

Now a process of separately using a combination of the angiography apparatus 11 and first cradle 15 and a combination of the X-ray CT apparatus 13 and second cradle 17 will be described with reference to FIGS. 7 and 8.

FIG. 7 is a schematic top view generally showing the radiation therapeutic imaging system 1 in accordance with the present embodiment, and FIG. 8 is a schematic side view showing the radiation therapeutic imaging system 1 in accordance with the present embodiment.

As shown in FIGS. 7 and 8, the angiography apparatus 11 is disposed near the first cradle 15 in the radiation therapeutic imaging system 1. The X-ray CT apparatus 13 is disposed near the second cradle 17. Moreover, the angiography apparatus 11 and first cradle 15, and the X-ray CT apparatus 13 and second cradle 17 are separated by the door.

Such a condition in which the combination of the angiography apparatus 11 and first cradle 15 and the combination of the X-ray CT apparatus 13 and second cradle 17 are separately used is sometimes referred to as the second state.

Moreover, the door 19 is closed. To prevent radiation transmission, the interior of the door 19 is made of lead and its surface is covered with aluminum, for example.

The operator selects the second state at the mode selecting section 22. Moreover, the second monitoring section 32 is selected at the X-ray CT apparatus selecting section 26.

In the angiography room separated off by the door 19, the first monitoring section 30 monitors the relative position of the angiography apparatus 11 and first cradle 15. After adjusting the relative position, the angiography apparatus 11 conducts vascular imaging on a predefined region in a subject on the first cradle 15.

In the CT room separated off by the door 19, the second monitoring section 32 monitors the relative position of the X-ray CT apparatus 13 and second cradle 17. After adjusting the relative position, the X-ray CT apparatus 13 conducts tomographic imaging while moving on a predefined region in a subject on the second cradle 17.

At that time, the control apparatus 20 need not monitor the angiography apparatus 11 and X-ray CT apparatus 13 for preventing their collision, and the apparatuses are independently monitored.

Moreover, since the door 19 is closed, the angiography apparatus 11 and X-ray CT apparatus 13 need not be prohibited from simultaneously emitting X-rays.

Furthermore, the angiography apparatus 11 and X-ray CT apparatus 13 are set to be independently stopped in an emergency.

Now a process of shifting from the second state to the first state will be described with reference to FIG. 1 and FIGS. 9-11.

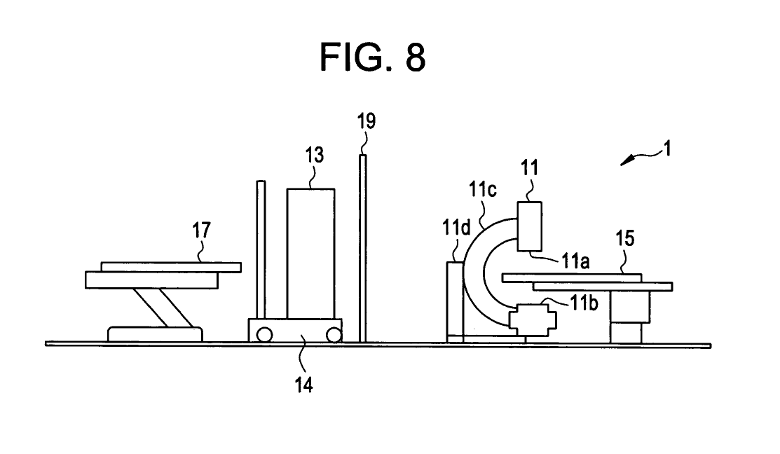

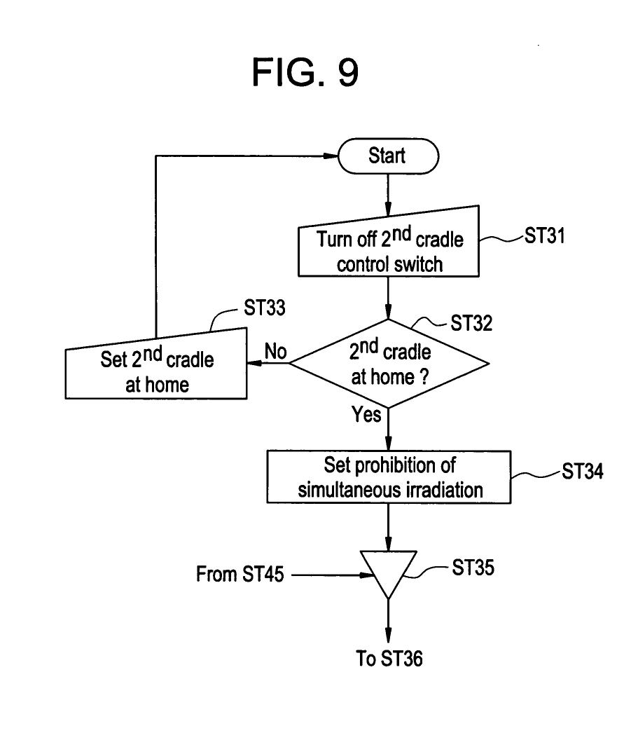

FIGS. 9-11 are flow charts generally showing the process of shifting from the second state to the first state in the radiation therapeutic imaging system in accordance with the present embodiment.

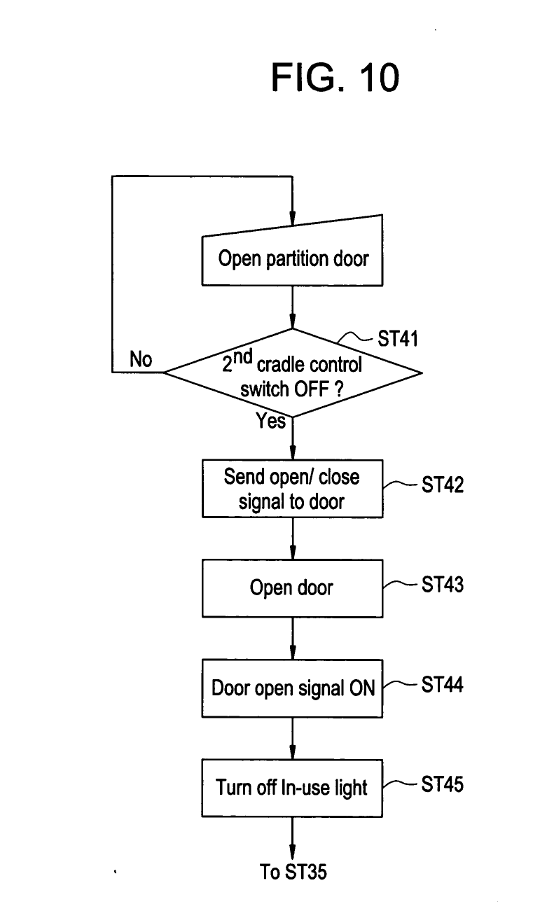

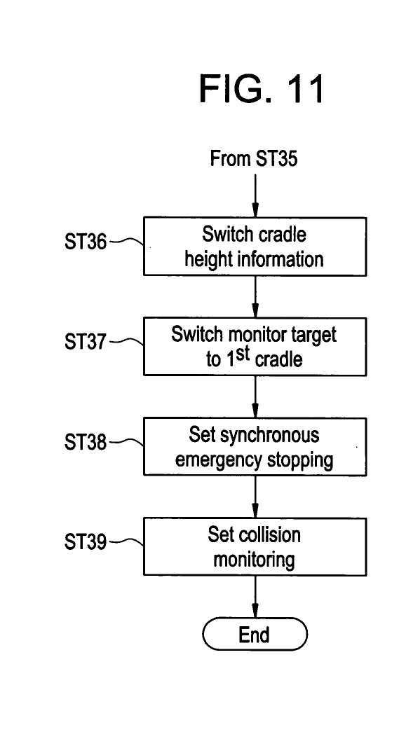

Steps ST31-ST39 shown in FIGS. 9 and 11 are mainly executed by the control apparatus 20, angiography apparatus driving/stopping apparatus 40 and X-ray CT apparatus driving/stopping apparatus 50 shown in FIG. 1, and Steps ST41-ST45 shown in FIG. 10 are mainly executed by the door opening/closing apparatus 34.

First, as shown in FIG. 9, the operator turns off the second cradle control switch 18 to exclude the second cradle 17 from control (ST31). Subsequently, the control apparatus 20 makes a confirmation that the second cradle 17 is at a predefined reference position (ST32). If the second cradle 17 is at a position different from the reference position, it is re-disposed at the reference position, and the process restarts with Step ST31 (ST33).

The control apparatus 20 next prohibits overlap of radiation emission by the angiography apparatus 11 and X-ray CT apparatus 13 (ST34). That is, the control apparatus 20 sets simultaneous irradiation prohibition.

On the other hand, as shown in FIG. 10, if one tries to open the door 19, the door opening/closing apparatus 34 first makes a confirmation that the second cradle control switch 18 is turned off via the door monitoring section 28 and mode selecting section 22 (ST41). If the switch 18 is not turned off, the flow of operations cannot proceed.

After making the confirmation that the second cradle control switch is turned off, the door opening/closing apparatus 34 sends an open/close signal to the door 19 (ST42). As a result, the door monitoring section 28 permits the door 19 to be opened, and the door 19 is opened (ST43). Upon opening of the door 19, a signal indicating the door 19 is open is output (ST44).

Next, the lights indicating that the two rooms separated by the door 19 are in use are turned off (ST45).

When the two conditions that the simultaneous irradiation prohibition is set (ST34), and that the lights indicating usage are turned off (ST45) have been confirmed (ST35) in FIG. 9, the height of the first cradle 15 is adjusted to be suitable for imaging using the angiography apparatus 11 and X-ray CT apparatus 13, as shown in FIG. 11 (ST36).

Next, the X-ray CT apparatus selecting section 26 switches monitoring of the X-ray CT apparatus 13 from the second monitoring section 32 to the first monitoring section 30. As a result, the first monitoring section 30 is set to monitor the relative position of the X-ray CT apparatus 13 and first cradle 15 (ST37).

Next, since the angiography apparatus 11 and X-ray CT apparatus 13 have been set to be independently stopped in an emergency in the second state, the control apparatus 20 sets them to synchronously stop in an emergency (ST38).

Next, the first monitoring section 30 is set to monitor the relative position of the angiography apparatus 11 and X-ray CT apparatus 13 (ST39).

By the process as described above, the second state is shifted to the first state.

While the angiography apparatus 11 as shown in FIG. 3 is used in the present embodiment, an angiography apparatus comprising a front arm for imaging the subject from the front and a side arm for imaging the subject from the side may be used.

Moreover, the angiography apparatus driving/stopping apparatus 40, X-ray CT apparatus driving/stopping apparatus 50, control apparatus 20 and door opening/closing apparatus 34 etc. may be installed in a room next to the room in which the therapeutic imaging system of the present embodiment is installed, so that the operator can operate the apparatuses together.

According to the radiation therapeutic imaging system of the present embodiment, the distance of travel of the X-ray CT apparatus 13 and base 14 is reduced when shifting from the first state to the second state, thereby shortening the time for switching between the first and second states. As a result, the availability rates of the apparatuses are improved. Moreover, since the X-ray CT apparatus 13 and base 14 are moved on a straight line, safety securance in the travel direction is facilitated. Furthermore, since the distance of travel is reduced, the wiring cables attached to the X-ray CT apparatus 13 are shortened as compared with conventional ones. In the radiation therapeutic imaging system in accordance with the present embodiment, imaging can be conducted in an elongated room so long as it has enough room to accommodate the X-ray CT apparatus, angiography apparatus and cradles.

Moreover, when the angiography apparatus 11 and X-ray CT apparatus 13 are separately used, an operation such as positioning can be easily conducted because the first cradle 15 and second cradle are adapted for the respective apparatuses.

Furthermore, imaging by the X-ray CT apparatus 13 can be conducted while moving in a direction coinciding with the direction of travel of the base 14.

### SECOND EMBODIMENT

A second embodiment in accordance with the present invention will now be described with reference to the accompanying drawings. Description on similar portions to those in the first embodiment designated by similar reference numerals will be omitted, and only different portions will be described.

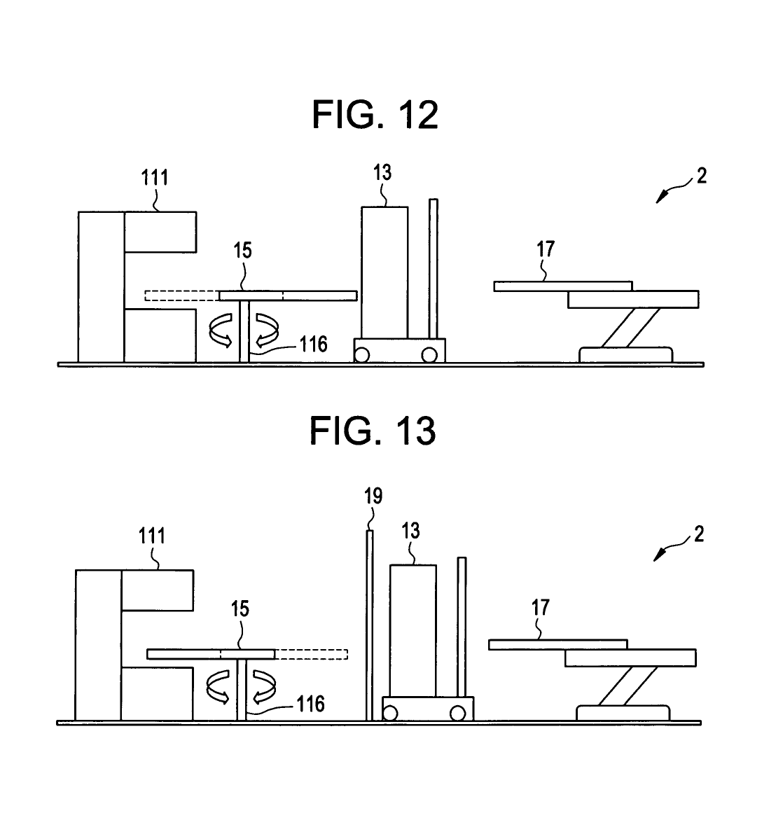

FIG. 12 is a schematic side view generally showing a radiation therapeutic imaging system 2 in accordance with the present embodiment, wherein the first state is illustrated in which IVR is conducted using the X-ray CT apparatus and radiation therapeutic apparatus in combination.

The radiation therapeutic imaging system 2 in accordance with the present embodiment comprises a radiation therapeutic apparatus 111, the X-ray CT apparatus 13, first cradle 15 and second cradle 17. The first and second cradles 15 and 17 are concatenately disposed so that their longitudinal dimensions are aligned on a straight line. Although not shown, a rail is provided between the first and second cradles 15 and 17 for guiding the X-ray CT apparatus 13.

Moreover, the first cradle 15 comprises a post 116 for supporting from the floor a table for laying thereon the subject. Furthermore, although not shown, the open/close door 19 is provided between the first and second cradles 15 and 17.

The radiation therapeutic apparatus 111 performs therapy by irradiating a predefined detected region such as cancerous cells with radiation.

One embodiment of the first radiation therapeutic/imaging apparatus of the present invention corresponds to the radiation therapeutic apparatus 111, and one embodiment of the second radiation therapeutic/imaging apparatus corresponds to the X-ray CT apparatus 13.

The radiation therapeutic imaging system 2 in accordance with the present embodiment first conducts tomographic imaging by the X-ray CT apparatus 13 for locating a position to be irradiated with radiation, and the position of the detected region is confirmed. Data of the detected region located by the tomographic imaging in the x-, y- and z-directions are transferred to the radiation therapeutic apparatus 111.

After completing the imaging by the X-ray CT apparatus 13, the first cradle 15 bearing thereon the subject is rotated in a horizontal plane around the axis 116. At that time, the rotation is made so that a scan center of the radiation therapeutic apparatus 111 and an imaging center of the X-ray CT apparatus 13 substantially coincide.

In such a condition, the radiation therapeutic apparatus 111 performs is therapy on the predefined position in the subject based on the transferred data. As in the first embodiment, the relative position of the radiation therapeutic apparatus 111, X-ray CT apparatus 13 and first cradle 15 is controlled by the control apparatus 20.

In the first state, the operator opens the door 19 after completing therapy by the radiation therapeutic apparatus 111. Subsequently, the X-ray CT apparatus 13 is moved on a straight line to the vicinity of the second cradle 17. The steps therefor are substantially the same as those in the first embodiment. After the X-ray CT apparatus 13 has been moved, the operator closes the door 19 again.

FIG. 13 is a schematic side view generally showing the radiation therapeutic imaging system 2 in accordance with the present embodiment, wherein the second state is illustrated in which a combination of the radiation therapeutic apparatus 111 and first cradle 15 and a combination of the X-ray CT apparatus 13 and second cradle 17 are separately used.

In the radiation therapeutic imaging system 2 shown in FIG. 13, the X-ray CT apparatus 13 is disposed near the second cradle 17. Moreover, the door 19 for preventing radiation transmission is closed to separate a region having the radiation therapeutic apparatus 111 and first cradle 15 and a region having the X-ray CT apparatus 13 and second cradle 17 by the door 19.

The door 19 is for preventing radiation transmission, and separating the radiation therapeutic apparatus 111 and X-ray CT apparatus 13.

In such a condition, since the X-ray CT apparatus 13 and radiation therapeutic apparatus 111 are separated by the door 19 for preventing radiation transmission, they can be used in a state of overlapping radiation exposure. That is, the radiation therapeutic apparatus 111 and X-ray CT apparatus 13 separated by the door 19 can be simultaneously used.

The operation and control method of the radiation therapeutic imaging system 2 in accordance with the present embodiment are substantially the same as those of the radiation therapeutic imaging system 1 in the first embodiment.

According to the radiation therapeutic imaging system of the present embodiment, the distance over which the X-ray CT apparatus 13 and base 14 are moved is reduced when shifting from the first state to the second state, thereby shortening the switching time. As a result, the availability rates of the apparatuses are improved. Moreover, since the X-ray CT apparatus 13 and base 14 are moved on a straight line, safety securance in the travel direction is facilitated. Furthermore, the reduced distance of travel allows the wiring cables in the X-ray CT apparatus 13 to be shortened as compared with conventional ones. In the radiation therapeutic imaging system in accordance with the present embodiment, imaging can be conducted in an elongated room so long as it has enough room to accommodate the X-ray CT apparatus, radiation therapeutic apparatus and cradles.

### THIRD EMBODIMENT

A third embodiment in accordance with the present invention will now be described with reference to the accompanying drawings. Description on similar portions to those in the first and second embodiments designated by similar reference numerals will be omitted, and only different portions will be described.

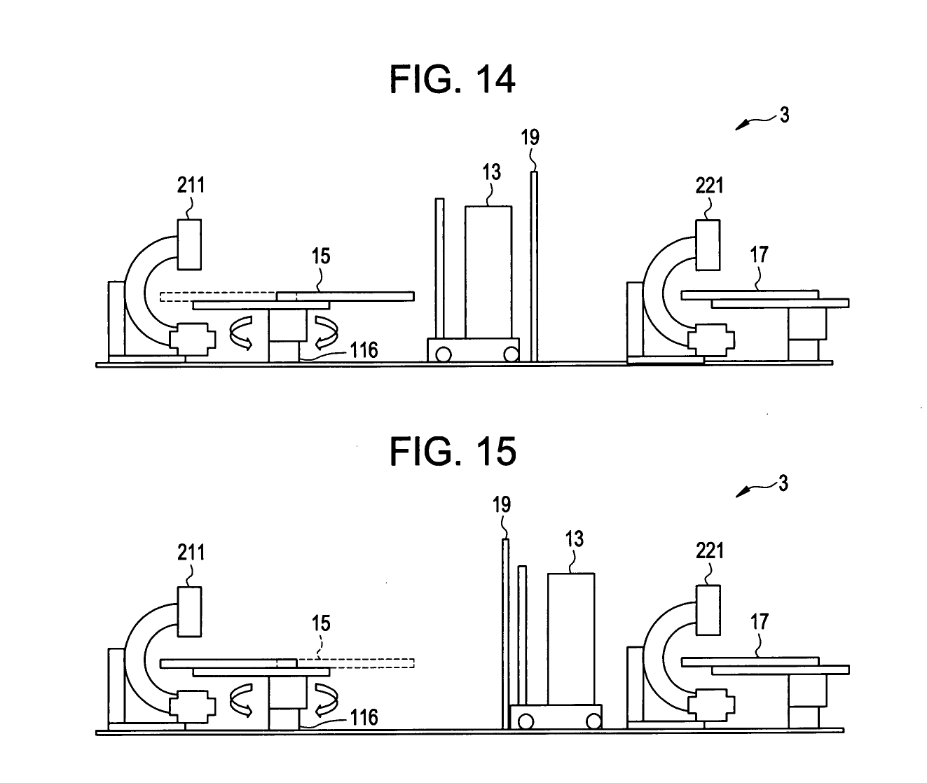

FIG. 14 is a schematic side view generally showing a radiation therapeutic imaging system 3 in accordance with the present embodiment.

The radiation therapeutic imaging system 3 in accordance with the present embodiment comprises a first angiography apparatus 211, the X-ray CT apparatus 13, first cradle 15, second cradle 17, and a second angiography apparatus 221. The door 19 is provided between the first and second cradles 15 and 17.

The first and second angiography apparatuses 211 and 221 conduct vascular imaging using X-rays as radiation.

One embodiment of the first radiation therapeutic/imaging apparatus of the present invention corresponds to the first angiography apparatus 211. One embodiment of the second radiation therapeutic/imaging apparatus of the present invention corresponds to the X-ray CT apparatus 13. Moreover, one embodiment of the third radiation therapeutic/imaging apparatus of the present invention corresponds to the second angiography apparatus 221.

In the present embodiment, since the first and second angiography apparatuses 211 and 221 are disposed near the first and second cradles 15 and 17, respectively, IVR can be conducted at both the first and second cradles 15 and 17 by moving the X-ray CT apparatus 13.

Therefore, a state in which the first angiography apparatus 211 and X-ray CT apparatus 13 are used in combination will be referred to as the first state, and a state in which the second angiography apparatus 221 and X-ray CT apparatus 13 are used in combination will be referred to as the second state hereinbelow.

The radiation therapeutic imaging system 3 shown in FIG. 14 conducts vascular imaging in the first state using the first angiography apparatus 211, and then conducts tomographic imaging using the X-ray CT apparatus 13 after rotating the first cradle 15. At that time, the first angiography apparatus 211, X-ray CT apparatus 13 and first cradle 15, and the second angiography apparatus 221 and second cradle 17 are separated by the door 19. Moreover, the second angiography apparatus 221 and second cradle 17 can independently conduct vascular imaging.

At that time, the X-ray CT apparatus 13 is monitored so that its position relative to the first cradle 15 is controlled.

Moreover, overlap of radiation emission by the first angiography apparatus 211 and X-ray CT apparatus 13 is prohibited, and they are controlled separately from the second angiography apparatus 211.

In the first state, after tomographic imaging by the X-ray CT apparatus 13 has been completed, the operator opens the door 19. Subsequently, the X-ray CT apparatus 13 is moved to the vicinity of the second cradle 17 along a rail on a straight line connecting the first and second cradles 15 and 17. The steps therefor are substantially the same as those in the first embodiment. After the X-ray CT apparatus 13 has been moved, the operator closes the door 19 again.

FIG. 15 is a schematic side view generally showing the radiation therapeutic imaging system 3 in accordance with the present embodiment, wherein the second state is illustrated.

In the radiation therapeutic imaging system 3 shown in FIG. 15, the X-ray CT apparatus 13 is moved to the vicinity of the second cradle 17. Moreover, the door 19 for preventing radiation transmission is closed to separate a region having the first angiography apparatus 211 and first cradle 15 and a region having the second angiography apparatus 221, X-ray CT apparatus 13 and second cradle 17 by the door 19.

At that time, overlap of radiation emission by the second angiography apparatus 221 and X-ray CT apparatus 13 is prohibited, and they are controlled separately from the first angiography apparatus 211.

The operation and control method of the radiation therapeutic imaging system 3 in accordance with the present embodiment are substantially the same as those of the radiation therapeutic imaging system 1 in the first embodiment.

According to the radiation therapeutic imaging system of the present embodiment, the distance over which the X-ray CT apparatus 13 and base 14 are moved is reduced when shifting from the first state to the second state, thereby shortening the switching time. As a result, the availability rates of the apparatuses are improved. Moreover, since the X-ray CT apparatus 13 and base 14 are moved on a straight line, safety securance in the travel direction is facilitated. Furthermore, the reduced distance of travel allows the wiring cables in the X-ray CT apparatus 13 to be shortened as compared with conventional ones. In the radiation therapeutic imaging system in accordance with the present embodiment, imaging can be conducted in an elongated room so long as it has enough room to accommodate the X-ray CT apparatus, radiation therapeutic apparatuses and cradles.

The therapeutic imaging system, and radiation therapeutic imaging system and method of controlling it of the present invention are not limited to those in the embodiments set forth above.

For example, an X-ray CT apparatus was specifically described as the radiation therapeutic/imaging apparatus of the present invention, but the present invention may be applied to other radiation therapeutic apparatuses or radiation imaging apparatuses. Moreover, other imaging apparatuses, such as an MRI (magnetic resonance imaging) apparatus, may be used as well as the radiation therapeutic/imaging apparatus.

In addition, various other modifications may be made without departing from the spirit and scope of the present invention.

Many widely different embodiments of the invention may be configured without departing from the spirit and the scope of the present invention. It should be understood that the present invention is not limited to the specific embodiments described in the specification, except as defined in the appended claims.

1. A therapeutic imaging system comprising:
a first therapeutic/imaging apparatus for performing at least one of therapy and imaging;
a second therapeutic/imaging apparatus for use in combination with said first therapeutic/imaging apparatus for performing at least one of therapy and imaging;
a first carriage device for laying thereon a subject, for use in combination with at least one of said first and second therapeutic/imaging apparatuses;
a second carriage device for laying thereon a subject, for use in combination with said second therapeutic/imaging apparatus, spaced apart from said first carriage device at a certain distance; and
a moving device disposed between said first and second carriage devices,
wherein said first and second carriage devices are concatenately disposed, and
said moving device moves said second therapeutic/imaging apparatus on a straight line connecting said first and second carriage devices.

2. The therapeutic imaging system of claim 1, further comprising:
an open/close door provided between said first and second carriage devices for separating a region in which said first and second carriage devices are installed into two sub-regions.

3. The therapeutic imaging system of claim 1, wherein:
said first therapeutic/imaging apparatus, said second therapeutic/imaging apparatus, said first carriage device and said second carriage device are moved relative to one another; and
said therapeutic imaging system further comprises a control device for selecting a combination of monitor targets according to a state of usage, and monitoring and controlling the relative position in the selected combination.

4. The therapeutic imaging system of claim 1, further comprising:
a third therapeutic/imaging apparatus disposed near said second carriage device for use in combination with said second therapeutic/imaging apparatus, for performing at least one of therapy and imaging.

5. A radiation therapeutic imaging system comprising:
a first radiation therapeutic/imaging apparatus for performing at least one of therapy and imaging by emitting radiation;
a second radiation therapeutic/imaging apparatus for use in combination with said first radiation therapeutic/imaging apparatus for performing at least, one of therapy and imaging by emitting radiation;
a first carriage device for laying thereon a subject, for use in at least one of said first and second radiation therapeutic/imaging apparatuses;
a second carriage device for laying thereon a subject, for use in combination with said second radiation therapeutic/imaging apparatus, spaced apart from said first carriage device at a certain distance;
a moving device disposed between said first and second carriage devices; and
an open/close door provided between said first and second carriage devices for separating a region in which said first and second carriage devices are installed into two sub-regions,
wherein said first and second carriage devices are concatenately disposed, and
said moving device moves said second radiation therapeutic/imaging apparatus on a straight line connecting said first and second carriage devices.

6. The radiation therapeutic imaging system of claim 5, wherein:
said door prevents radiation transmission.

7. The radiation therapeutic imaging system of claim 5, wherein:
said first radiation therapeutic/imaging apparatus, said second radiation therapeutic/imaging apparatus, said first carriage device and said second carriage device are moved relative to one another; and
said radiation therapeutic imaging system further comprises a control device for changing a combination of monitor targets according to a state of usage, and monitoring and controlling the relative position in the combination.

8. The radiation therapeutic imaging system of claim 7, wherein:
said control device prohibits or permits overlap of radiation emission by said first and second radiation therapeutic/imaging apparatuses according to a state of usage.

9. The radiation therapeutic imaging system of claim 7, further comprising:
first and second driving/stopping devices for driving and emergency-stopping said first and second radiation therapeutic/imaging apparatuses;
wherein said first and second driving/stopping devices are controlled by said control device to synchronously or separately emergency-stop said first and second radiation therapeutic/imaging apparatuses according to a state of usage.

10. The radiation therapeutic imaging system of claim 5, further comprising:
a third radiation therapeutic/imaging apparatus disposed near said second carriage device for use in combination with said second radiation therapeutic/imaging apparatus.

11. The radiation therapeutic imaging system of claim 1, wherein:
at least one of said first and second carriage devices is rotatable in a horizontal plane of said first or second carriage device.

12. The radiation therapeutic imaging system of claim 5, wherein:
said second radiation therapeutic/imaging apparatus is comprised of a radiation tomographic imaging apparatus.

13. The radiation therapeutic imaging system of claim 12, wherein:
said radiation tomographic imaging apparatus conducts imaging while moving.

14. The radiation therapeutic imaging system of claim 5, wherein:
said first radiation therapeutic/imaging apparatus is comprised of either a radiation tomographic imaging apparatus, a radiation therapeutic apparatus or a vascular imaging apparatus.

15. The radiation therapeutic imaging system of claim 5, wherein:
said third radiation therapeutic/imaging apparatus is comprised of either a radiation tomographic imaging apparatus, a radiation therapeutic apparatus or a vascular imaging apparatus.

16. A method of controlling a radiation therapeutic imaging system for use in a radiation imaging system comprising: a first radiation therapeutic/imaging apparatus for performing at least one of therapy and imaging by radiation; a second radiation therapeutic/imaging apparatus that is movable, for use in combination with said first radiation therapeutic/imaging apparatus for performing at least one of therapy and imaging by radiation; a first carriage device for laying thereon a subject, for use in at least one of said first and second radiation therapeutic/imaging apparatuses; a second carriage device for laying thereon a subject, for use in combination with said second radiation therapeutic/imaging apparatus, spaced apart from said first carriage device at a certain distance and disposed concatenately with said first carriage device; a moving device disposed between said first and second carriage devices for moving said second therapeutic/imaging apparatus on a straight line connecting said first and second carriage devices; and an open/close door provided between said first and second carriage devices for separating a region in which said first and second carriage devices are installed into two sub-regions, said method comprising:
moving said first radiation therapeutic/imaging apparatus, said second radiation therapeutic/imaging apparatus, said first carriage device and said second carriage device relative to one another; and
said method comprising the steps of, in a first condition,:
performing therapy or imaging at a predefined position by said second radiation therapeutic/imaging apparatus with said first radiation therapeutic/imaging apparatus fixed, while monitoring positions of at least said second radiation therapeutic/imaging apparatus and said first carriage device; and
performing therapy or imaging at a predefined position by said first radiation therapeutic/imaging apparatus with said second radiation therapeutic/imaging apparatus fixed, while monitoring positions of at least said first radiation therapeutic/imaging apparatus and said first carriage device; and
in a second condition, performing therapy or imaging at a predefined position by said first radiation therapeutic/imaging apparatus while monitoring positions of said first radiation therapeutic/imaging apparatus and said first carriage device; and
performing therapy or imaging at a predefined position by said second radiation therapeutic/imaging apparatus while monitoring positions of said second radiation therapeutic/imaging apparatus and said second carriage device,
wherein in said second condition, said method separately conducts the step of performing therapy or imaging by said first radiation therapeutic/imaging apparatus and the step of performing therapy or imaging by said second radiation therapeutic/imaging apparatus.

17. The radiation therapeutic imaging system of claim 5, wherein:
at least one of said first and second carriage devices is rotatable in a horizontal plane of said first or second carriage device.
